(19) United States
(12) Patent Application Publication
(10) Pub. No.: US 2026/0056178 A1
(43) Pub. Date: Feb. 26, 2026

(54) OPTICALLY ACCESSIBLE SYSTEM FOR STUDIES ON EMBRYONATED EGGS

(71) Applicant: POLITECNICO DI MILANO, Milano (MI) (IT)

(72) Inventors: Manuela Teresa RAIMONDI, Milano (MI) (IT); Claudio CONCI, San Donato Milanese (MI) (IT); Leonardo CHERUBIN, Clivio (VA) (IT); Bianca BARZAGHINI, Milano (MI) (IT); Matteo LAGANA, LONGONE AL SEGRINO (CO) (IT)

(21) Appl. No.: 19/112,001

(22) PCT Filed: Sep. 8, 2023
(86) PCT No.: PCT/IB2023/058925

§ 371 Date: Mar. 14, 2025

(30) Foreign Application Priority Data
Sep. 14, 2022 (IT) 102022000018789

(51) Int. Cl. G01N 33/08

(52) U.S. Cl. CPC G01N 33/08

(57) ABSTRACT

An optically accessible system for studies on embryonated eggs characterized in that it comprises a disc (10) having a concave inner surface to reflect the profile of an egg; said disc (10) comprises a central through hole (11); said hole (11) is closed by a transparent plate (16) to make said system optically accessible; said through hole (11) is adapted to be placed alongside the respiratory membrane of an embryonated egg from which, in the portion where said system is to be made optically accessible, the shell has been partially removed.

### DESCRIPTION

The present invention refers to an optically accessible system for studies on embryonated eggs and relative production method.

The development of new drugs or vaccines is a process that takes time and entails high costs. The striking impact on society of the Sars-Cov-2 pandemic has highlighted the need for in vivo experimental models to accelerate development and reduce risks for the patient, reduce costs and the ethical impact of the preclinical validation of new biologic or antiviral drugs, such as genetically modified cells, small inhibitory molecules, growth factors, antibodies, RNA-messengers etc. This need to reduce the risks, times, costs and ethical impact relative to development and pharmacological validation is particularly urgent for drugs that act on the microcirculation (for example, in diabetes, diseases of the retina, in kidney diseases, in virology, etc.) In the same way, the vascular network is the target in the development of antiangiogenic drugs, based on antibodies and small molecules in cancer research.

The object of the present invention is to provide an optically accessible system to allow intravital studies on embryonated eggs which is simple, stable and minimally invasive.

A further object is to provide a system that is effective. In accordance with the present invention, said objects and others are achieved by an optically accessible system for studies on embryonated eggs characterized in that it comprises a disc having a concave inner surface to reflect the profile of an egg; said disc comprises a central through hole; said hole is closed by a transparent plate to make said system optically accessible; said through hole is adapted to be placed alongside the respiratory membrane of an embryonated egg from which, in the portion where said system is to be made optically accessible, the shell has been partially removed.

Said objects are furthermore achieved by a method for producing an optically accessible system for studies on embryonated eggs in accordance with claim 1 comprising the steps of removing at least a portion of shell from an egg; and placing said through hole alongside the respiratory membrane of an embryonated egg.

Further characteristics of the invention are described in the dependent claims.

This solution offers various advantages with respect to the solutions of the known art.

The system is a synthetic device that allows the vascular capillary tissue of the chorioallantoic membrane of an embryonated poultry egg to be viewed at different time instants, preferably by means of optical microscopes, simultaneously ensuring sterility during the entire observation process and allowing the creation of intravital bioengineered models that can be used in any biological laboratory.

The system allows pre-screening of the effect of the therapeutic agents on the microvascular network with a rapid, versatile and real-time approach that can be used in the pharmaceutical industries, in research laboratories, in contract research organizations and in other lifescience sectors.

The device can be applied directly on the physiological poultry egg shell or combined with a specific synthetic shell substitute. The device can be loaded with 3D microenvironments, like scaffolds for cell cultures (for example, hydrogels or microgrids functionalized and/or seeded with cell models) or with miniaturized imaging windows.

The system allows in vivo analyses to be performed in an autonomous compact device that has lower correlated costs. Compared to other market alternatives, such as genetically modified laboratory animals, the system will allow a new approach to in vivo studies. Compared to lab-on-a-chip platforms, it comprises a highly engineered imaging window and a living embryonal model that provides data relative to an interaction with a developing organism. The system can clearly replace the lab-on-a-chip models and does not require authorization for animal testing, allowing its rapid use in a wide variety of biosafety level 2 (BSL-2) laboratories and application cases. In fact, animal testing facilities are not necessary, drastically reducing staff and service costs. Due to its embryonal nature, it allows rapid kinetic tests to be performed that ensure a narrower range of data collected, thus providing a rapid reliable instrument for molecule pre-screening before in vivo testing campaigns on adult animals.

The characteristics and advantages of the present invention will be evident from the following detailed disclosure of a practical embodiment thereof, illustrated by way of non-limiting example in the attached drawings, in which:

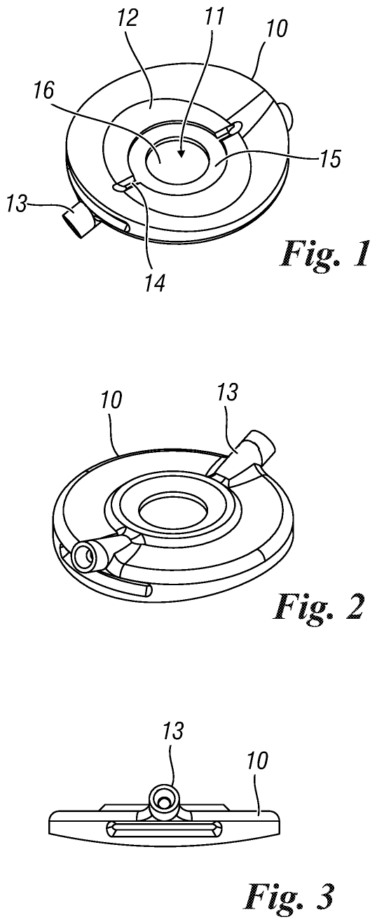

FIG. 1 shows a disc for an optically accessible system for studies on embryonated eggs, seen from below, in accordance with the present invention;

FIG. 2 shows a disc for an optically accessible system for studies on embryonated eggs, seen from above, in accordance with the present invention;

FIG. 3 shows a disc for an optically accessible system for studies on embryonated eggs, seen from the side, in accordance with the present invention;

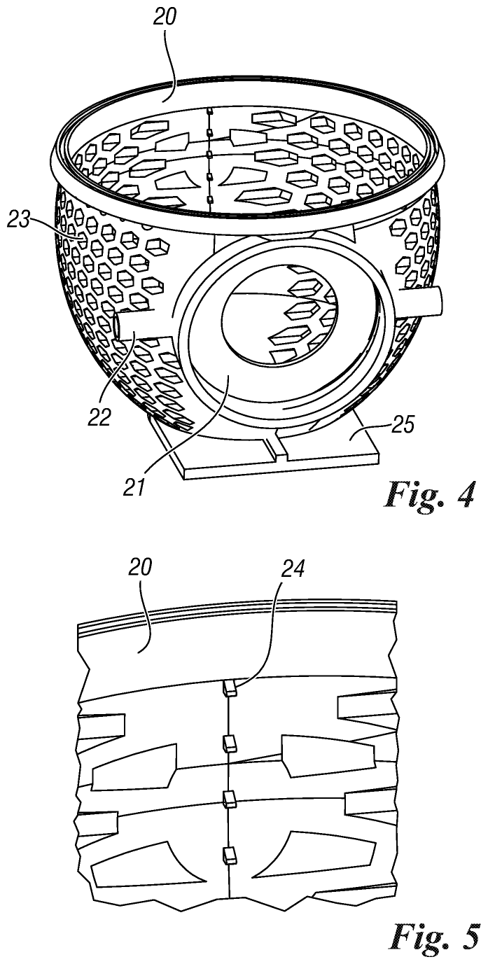

FIG. 4 shows a body of an optically accessible system for studies on embryonated eggs, in accordance with the present invention;

FIG. 5 shows an enlargement of a body of an optically accessible system for studies on embryonated eggs, in accordance with the present invention;

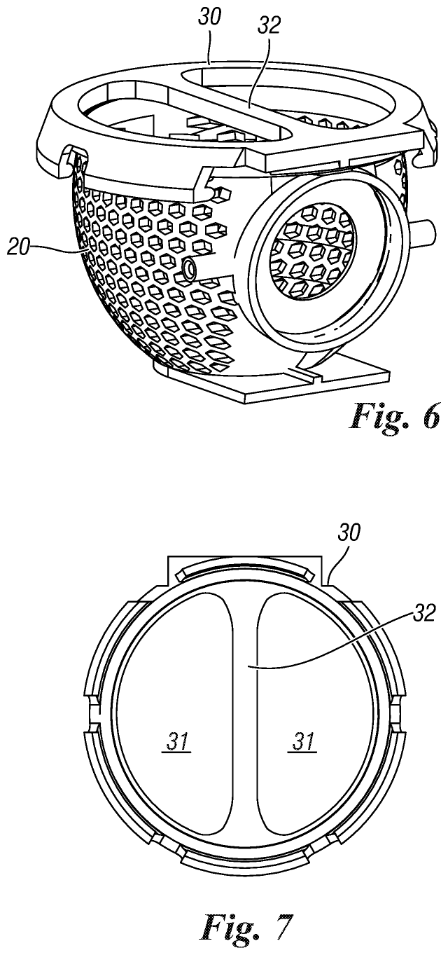

FIG. 6 shows a body of an optically accessible system for studies on embryonated eggs, including an upper closing crown, in accordance with the present invention;

FIG. 7 shows an upper crown of an optically accessible system for studies on embryonated eggs, seen from below, in accordance with the present invention;

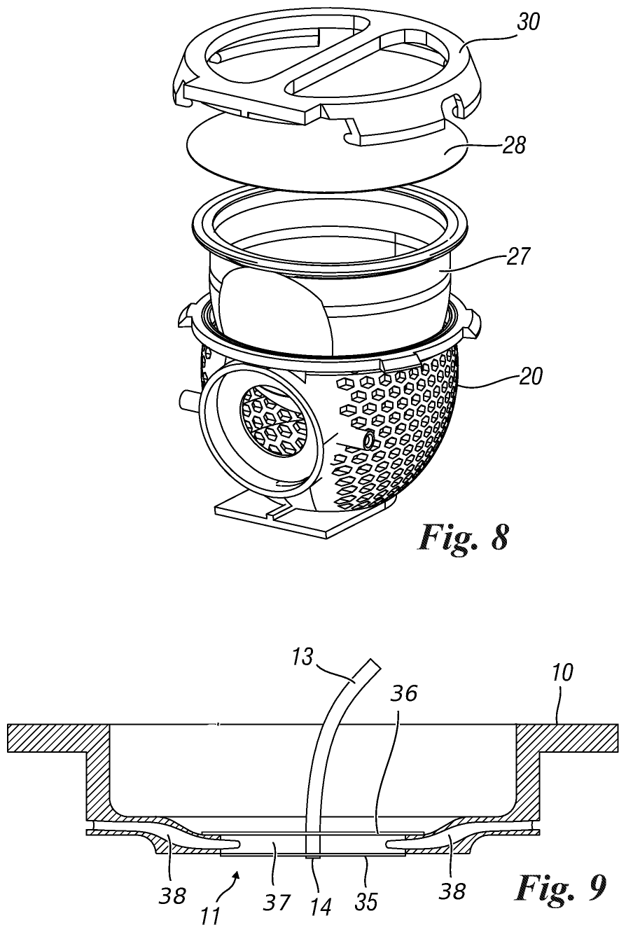

FIG. 8 shows a body of an optically accessible system for studies on embryonated eggs, including a breathable membrane, in accordance with the present invention;

FIG. 9 shows a disc for an optically accessible system for studies on embryonated eggs, seen in section, in accordance with a variation of the present invention.

Referring to the attached figures, an optically accessible system for studies on embryonated eggs, in accordance with the present invention, comprises a disc 10 having the function of a plug for an egg from which at least a part of the shell has been removed.

The disc 10 has a circular shape with a concave inner surface to reflect the profile of an egg, and comprises a central through hole 11 and a first edge 12 sunken relative to the inner surface of the disc 10, circular and coaxial to the hole 11, and a second edge 15, sunken relative to the first edge 12, circular and coaxial to the first edge 12.

A transparent plate 16, for example a slide or another optically accessible means, is applied to the second edge 15, for example by gluing in the case of glass or by suitable coupling method, such as hot or ultrasonic welding, in the case of plastic inserts.

On the first edge 12 it is possible to apply circular membranes 17 or slides or coverslips or biomaterials incised with a circular geometry with compatible diameter. The disc 10 furthermore has two tubular external inlets 13 positioned laterally to its outer surface and directed towards the hole 11, having indicative diameter from 0.5 to 3 mm, preferably 2 mm.

The two inlets 13 give access to the inner part of the disc 10 in the vicinity of the hole 11 with two microcapillary holes 14 having diameter of 0.5 mm.

These holes 14, facing the inside of the egg, allow the region exposed to direct observation to be perfused with a flow or allow the administration or intravital sampling of fluids to/from the poultry model without the need for invasive interventions. Said channels could also be used to introduce particular instruments adapted to withdraw portions of biological tissue, for example needles for biopsies or small laparoscopic instruments for microsurgery.

Considering by way of example eggs from a ROSS 308 chicken, the disc 10 has a diameter of at least 28 mm and a thickness of 3 mm. The through hole 11 has a diameter of at least 8 mm, the first edge 12 has a diameter of 12 mm and a depth of 1 mm, and the second edge 15 has a diameter of 10 mm and a depth of 1 mm.

The disc is made of a soft material such as, for example, NBR, Silicone, TPE, TPU, or a material permeable to medical gases (such as oxygen), circular, mouldable in terms of dimensions for optimal adaptation to the size of the chicken egg used.

The disc, thus formed, will also be able to compensate for the thickness of the body to which it is joined, bringing the coverslip to the same level as the inside of the egg. This technical solution therefore avoids the creation of stepped inserts that can lacerate the respiratory membrane of the chicken embryo once coupled. The disc also allows the housing of scaffolds, applied to the first edge 12, for cell cultures (which can be made of sol-gel, organic/inorganic synthetic materials etc.), transparent imaging windows or organ-on-chip models.

The disc 10 can be applied directly to the egg after removal of a part of the shell, or can be applied to a body 20 that completely replaces the eggshell, so that the outer surface of the disc 10 is positioned alongside the respiratory membrane of an embryonated egg.

The body 20 has the shape of an ellipsoidal vase, with a circular opening at the top for insertion of the egg without shell, such as to adapt to the shape of a medium-sized chicken egg, and is made of medical plastic such as, for example, polycarbonate, ABS, MABS, PP, PET, polysulfone, polystyrene, or other rubbery materials like NBR, Silicone, TPE or TPU.

In an embodiment example, the body 20 has an overall height of approximately 36 mm and a maximum width of approximately 50 mm.

The body 20 consists of a single block having a circular lateral inlet 21. The inlet 21 is sunken and has a depth such as to be coupled by mechanical interference with the disc 10. Laterally to the inlet 21 are tube portions 22 that can be connected to the inlets 13 of the disc 10.

The body 20 comprises a plurality of small windows 23 (through holes) which define the porosity of the body 20. The windows 23 preferably have a hexagonal shape and an area of approximately 3 mm² each and in any case less than 4 mm².

Different dimensions are possible for the windows 23 to modify the porosity or to increase or decrease the optical access capability or the gaseous exchange through the permeable wall.

The windows 23 are sized so as to allow gaseous exchange and at the same time contain the shell-less egg without deforming it.

A breathable flexible membrane 27 that surrounds the egg is placed inside the body 20.

Inside the body 20 there are spacers 24, uniformly distributed, having rectangular section with side of 1 mm, which allow the breathable membrane 27 to be maintained detached from the wall of the body 20, thus maximizing the gaseous exchange, avoiding the membrane 27 coming into direct contact with the lateral walls of the body 20, thus creating areas without gaseous flow.

The breathable membrane 27 can be made for example of materials able to guarantee sterility but at the same time allow the transport of medical gases, for example oxygen. These can be indicatively, but without limitation, silicones (such as, for example, siloxanes) or synthetic fabrics based on high density polyethylene fibres (such as, for example, Tyvek, Polywrap) or polyurethanes (such as Tegaderm) or any other synthetic membrane, for example hydrogels, or natural membrane suitable for the purpose. This can have the shape of an ellipsoidal vase, with a circular opening at the top, such as to reflect the inner shape of the body 20, inclusive of shaping of the lateral inlet 21.

The membrane must be shapeable to obtain thicknesses of less than one millimetre, for example with a value of 0.1-0.5 mm. The membrane must guarantee as small as possible a difference in the gaseous concentration, for example in the case of oxygen, based on the poultry model positioned inside. For example, considering ROSS308 chicken eggs, the difference in the oxygen concentration, between outer and inner compartments of the membrane, must be no higher than 20% until the eighth day of incubation and lower than 60% until the twelfth day of incubation.

The body 20 has at the bottom a rectangular base 25 having dimensions 25 mm×40 mm which increases stability, allowing upright positioning of the body 20 once housed on a bench or inside an incubator for cell cultures.

The body 20 further comprises a circular upper closing crown 30, preferably divided by a central partition 32 into two symmetrical through sections 31.

The crown 30 is also made of medical plastic and closes the body.

The breathable membrane 27 can comprise an additional circular insert 28 which can guarantee the fixing of said membrane, for example by mechanical interference with the crown 30.

A breathable and/or oxygen-permeable insert is applied to the crown 30 by gluing, or by other suitable process, thus guaranteeing closing of the body 20.

The breathable closing insert can also be transparent, so as to provide optical access inside the body 20 also from above. Said crown 30 is joined to the body 20 at the top by means of a mechanical coupling or by means of threading.

The body 20 allows the chicken embryo to be moved in space, for example by inclining it on an optical bench or inside the incubation chamber of a microscope, in order to position the window (lateral through access) axially relative to the device, corresponding to the lens of the microscope system used, whether in straight, inverted or oblique configuration.

The disc 10 is applied to the lateral circular inlet 21 of the body 20 and an egg from which the shell has been completely removed is positioned inside the body 20.

In an embodiment of the present invention, the plug 10 has a shape slightly different from the previous plug as it is squarer and is used by applying it the opposite way round to previously, with the convex part towards the egg, instead of the concave part towards the egg.

The hole 11 of the plug 10 can be closed by an insert 35 applied to the outer surface of the plug 10, adapted to come into contact with the respiratory membrane of an embryonate egg; a slide 36 is positioned inside. The chamber 37 created between the slide 36 and the insert 35 can comprise two additional ducts 38 for the inlet and outlet of fluids or gases.

Said solution allows a flow of liquid or any medical gas to be created between the slide 36 and the insert 35, which can be a transparent synthetic or natural membrane, and can be structured with scaffolds for cell cultures or structures acting as beacons to obtain intravital images. The chamber 37 has a thickness of less than one millimetre.

1. An optically accessible system for studies on embryonated eggs characterized in that it comprises a disc (10) having a concave inner surface to reflect the profile of an egg; said disc (10) comprises a central through hole (11); said hole (11) is closed by a transparent plate (16) to make said system optically accessible; said through hole (11) is adapted to be placed alongside the respiratory membrane of an embryonated egg from which, in the portion where said system is to be made optically accessible, the shell has been partially removed.

2. The system according to claim 1 characterized in that said disc (10) comprises a first edge (12) which is circular and coaxial with the hole (11) to which said transparent plate (16) is applied.

3. The system according to claim 1 characterized in that said disc (10) comprises an outer surface and two tubular outer inlets (13) positioned laterally to said outer surface directed towards said hole (11).

4. The system according to claim 3 characterized in that said two inlets (13) give access to the inner surface of said disc (10) in the vicinity of said hole (11) with two holes (14).

5. The system according to claim 1 characterized in that said system comprises a body (20) having an ellipsoidal shape, open at the top, adapted to contain an egg without its shell; said body (20) comprises a circular lateral inlet (21); said disc (10) is adapted to be applied to close said lateral inlet (21).

6. The system according to claim 5 characterized in that said body (20) comprises a plurality of through holes (23) each having an area of less than 4 mm², which define the porosity of said body (20).

7. The system according to claim 6 characterized in that said body (20) comprises a breathable membrane, adapted to cover the porosity of said body (20) constituting a sterile barrier, permeable to gases but substantially impermeable to liquids.

8. The system according to claim 5 characterized in that said body (20) comprises an upper closing crown (30) to which a breathable insert is applied.

9. A method for producing an optically accessible system for studies on embryonated eggs in accordance with claim 1 comprising the steps of removing at least a portion of shell from an egg; and placing said through hole (11) alongside the respiratory membrane of an embryonated egg.
